(19) United States
(12) Patent Application Publication
(10) Pub. No.: US 2024/0033148 A1
(43) Pub. Date: Feb. 1, 2024

(54) WHEELCHAIR SECURABLE URINE COLLECTION SYSTEMS AND RELATED METHODS

(71) Applicant: PUREWICK CORPORATION, Covington, GA (US)

(72) Inventors: Tacarra Gordon, Loganville, GA (US); Sudhakar Jagannathan, Alpharetta, GA (US); Nicholas Jardine, Holly Springs, NC (US); Kuilin Lai, Watkinsville, GA (US); Puja Patel, Lawrenceville, GA (US)

(21) Appl. No.: 18/264,278

(22) PCT Filed: Feb. 9, 2022
(86) PCT No.: PCT/US2022/015781

§ 371 Date: Aug. 4, 2023

Related U.S. Application Data
(60) Provisional application No. 63/148,723, filed on Feb. 12, 2021.

(51) Int. Cl. A61G 5/10; A61F 5/451; A61F 5/44

(52) U.S. Cl. CPC A61G 5/1002; A61F 5/451; A61F 5/4404

(57) ABSTRACT

Examples relate to devices, systems, and methods for fluid collection such as urine. A portable urine collection system includes a urine collection device, a conduit in fluid communication with the urine collection device, a urine collection container having an interior region, a pump, and a container support configured to detachably secure to a motorized wheelchair that is adjustable between a sitting orientation and a standing orientation. The pump is configured to pull an at least partial vacuum on the interior region of the urine collection container effective to draw urine from the urine collection device through the conduit into the urine collection container when the motorized wheelchair is in each of the sitting orientation and the standing orientation. The container support is sized and dimensioned to support the urine collection container and the pump therein.

### CROSS-REFERENCE TO RELATED APPLICATIONS

This application claims priority to U.S. Provisional Patent Application No. 63/148,723 filed on Feb. 12, 2021, the disclosure of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections. Conventional urine collection devices also may be limited to use when a patient is confined to a bed in a supine position.

Thus, users and manufacturers of fluid collection devices continue to seek new and improved devices, systems, and methods to collect urine.

### SUMMARY

Embodiments disclosed herein are related to fluid collection devices and methods of using fluid collection devices. In an embodiment, a portable urine collection system is disclosed. The portable urine collection system includes a urine collection device configured to be positioned at least proximate to a urethra of a user, a conduit in fluid communication with the urine collection device, a urine collection container having an interior region, a pump in fluid communication with the urine collection container, and a container support configured to detachably secure to a motorized wheelchair that is adjustable between a sitting orientation, a standing orientation, and a reclined orientation. The pump is configured to pull an at least partial vacuum on the interior region of the urine collection container effective to draw urine from the urine collection device through the first conduit into the urine collection container when the motorized wheelchair in the sitting orientation, the standing orientation, and the reclined orientation. The container support is sized and dimensioned to support the urine collection container.

In an embodiment, a method of assembling a portable urine collection system is disclosed. The method includes detachably securing a container support to a motorized wheelchair that is adjustable between a sitting orientation, a standing orientation, and a reclined orientation. The method also includes inserting a urine collection container and a pump in fluid communication with an interior region of the urine collection container in the container support. The method also includes positioning a urine collection device proximate to a urethra of a user. The method also includes fluidly coupling the urine collection device to the urine collection container with a conduit. The pump is configured to pull an at least partial vacuum on the interior region of the urine collection container effective to draw urine from the urine collection device through the first conduit into the urine collection container and draw urine from the urine collection device when the motorized wheelchair is in each of the sitting orientation, the standing orientation, and the reclined orientation.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of a portable urine collection system, according to an embodiment.

FIG. 2 is a front view of a portable urine collection system, according to an embodiment.

FIG. 3A is a side view of a motorized wheelchair having a container support and in a sitting orientation, according to an embodiment.

FIG. 3B is a side view of a motorized wheelchair having a container support and in a standing orientation, according to an embodiment.

FIG. 4 is a flow diagram of a method for assembling a portable urine collection system, according to an embodiment.

FIG. 5 is a block diagram of a controller in a urine collection system, according to an embodiment.

### DETAILED DESCRIPTION

Embodiments disclosed herein are related to fluid collection devices and methods of using the same. The devices and systems disclosed herein are configured to collect fluids from an individual. The fluids collected by the fluid collection devices may include at least one of urine, vaginal discharge, penile discharge, reproductive fluids, blood, sweat, or other bodily fluids. Embodiments disclosed herein are related to wheelchair mountable urine collection systems and related methods. Many users of urine collection devices are over 65 years old with limited mobility, often relying on wheelchairs as a primary mode of transportation. Many users also spend a significant amount of their day in a seated or supine position. Users and caregivers, then, are benefited from a fluid collection system that may be both discrete and mobile, allowing users to use the urine collection system to collect urine both at home and on the go.

In at least one, some, or all of the embodiments described herein, a fluid collection system is compact and includes a configuration that allows the fluid collection system to be secured or securable (e.g., mounted or mountable) to a wheelchair. At least one, some, or all of the embodiments of the fluid collection systems described herein have a configuration resulting in the technical effect the fluid collection system being mobile and discreet, allowing a user to participate in social activities without alerting others to the incontinence of the users. In at least one, some, or all of the embodiments of fluid collection systems described herein, an alert system is included that is configured to communicate with an electronic device, resulting in the technical effect of alerting a user or caregiver to empty the fluid collection container when the fluid level approaches a predetermined level, to change or recharge a battery, and/or adjust a vacuum or suction level of the pump in the fluid collection system.

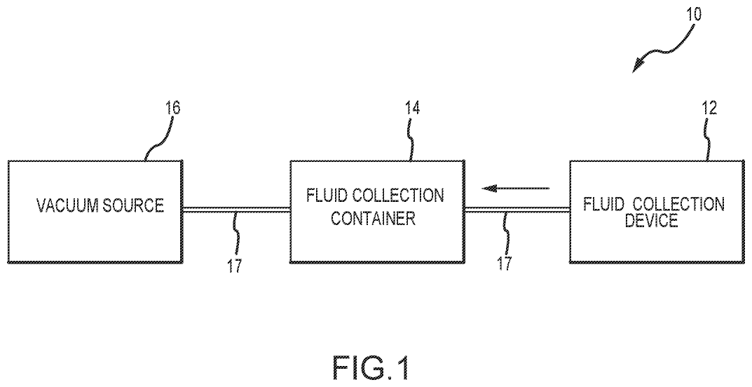

FIG. 1 is a block diagram of a fluid collection system 10, according to an embodiment. The fluid collection system 10 may be included in embodiments of fluid collection systems described herein. The system 10 includes a fluid collection device 12 (e.g., any of the fluid collection assemblies disclosed herein), a fluid collection container 14, and a vacuum source 16 (or pump). The fluid collection device 12, the fluid collection container 14, and the vacuum source 16 may be fluidly coupled to each other via one or more conduits 17. For example, fluid collection device 12 may be operably coupled to one or more of the fluid collection container 14 or the vacuum source 16 via the conduit 17. In some embodiments, the vacuum source 16 may be secured directly to the fluid collection container 14. Fluid (e.g., urine or other bodily fluids) collected in the fluid collection device 12 may be removed from the fluid collection device 12 via the conduit 17 secured to the fluid collection device 12. Suction force may be introduced into the chamber of the fluid collection device 12 via the inlet of the conduit 17 responsive to suction (e.g., vacuum) force applied at the outlet of the conduit 17.

The suction force may be applied to the outlet of the conduit 17 by the vacuum source 16 either directly or indirectly. The suction force may be applied indirectly via the fluid collection container 14. For example, the outlet of the conduit 17 may be disposed within or fluidly coupled to an interior region of the fluid collection container 14 and an additional conduit 17 may extend from the fluid collection container 14 to the vacuum source 16. Accordingly, the vacuum source 16 may apply suction to the fluid collection device 12 via the fluid collection container 14. The suction force may be applied directly via the vacuum source 16. For example, the outlet of the conduit 17 may be disposed within the vacuum source 16. An additional conduit 17 may extend from the vacuum source 16 to a point outside of the fluid collection device 12, such as to the fluid collection container 14. In such examples, the vacuum source 16 may be disposed between the fluid collection device 12 and the fluid collection container 14.

The fluid collection container 14 is sized and shaped to retain a fluid therein. The fluid collection container 14 may include a bag (e.g., drainage bag), a bottle or cup (e.g., collection jar), or any other enclosed container for storing bodily fluid(s) such as urine. In some examples, the conduit 17 may extend from the fluid collection device 12 and attach to the fluid collection container 14 at a first point therein. An additional conduit 17 may attach to the fluid collection container 14 at a second point thereon and may extend and attach to the pump 16. Accordingly, a vacuum (e.g., suction) may be drawn through fluid collection device 12 via the fluid collection container 14. Fluid, such as urine, may be drained from the fluid collection device 12 using the vacuum source 16.

The vacuum source 16 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 16 may provide a vacuum or suction to remove fluid from the fluid collection device 12. In some examples, the vacuum source 16 may be powered by one or more of a power cord (e.g., connected to a power socket), one or more batteries, or even manual power (e.g., a hand operated vacuum pump). In some examples, the vacuum source 16 may be sized and shaped to fit outside of, on, or within the fluid collection device 12. For example, the vacuum source 16 may include one or more miniaturized pumps or one or more micro pumps. The vacuum sources disclosed herein may include one or more of a switch, a button, a plug, a remote, or any other device suitable to activate the vacuum source 16.

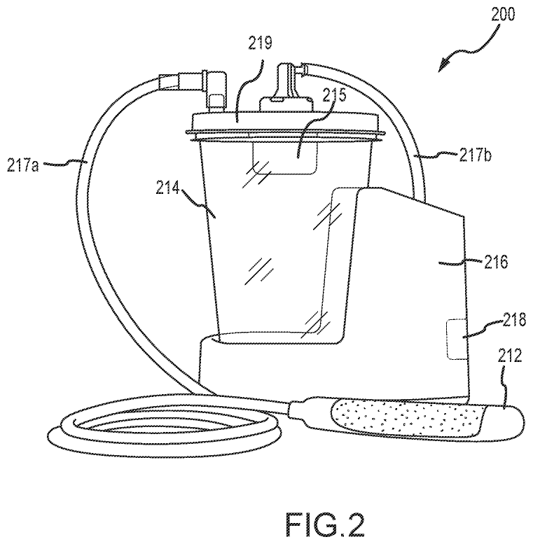

FIG. 2 shows an example of a urine collection system 200, according to an embodiment. While urine collection is referenced with respect to the urine collection system 200, the urine collection system 200 may collect other bodily fluids, such as vaginal discharge, penile discharge, reproductive fluids, blood, and/or sweat. The urine collection system 200 may include a urine collection device 212, a first conduit 217a in fluid communication with an interior region of the urine collection device 212, a urine collection container 214 having an interior region in fluid communication with the internal region of the urine collection device 212 via the first conduit 217a, a second conduit 217b in fluid communication with the interior region of the urine collection container 214, a pump 216 (e.g., vacuum source) in fluid communication with the interior region of the urine collection container 214 via the second conduit 217b , and a sensor 215. The urine collection device 212 is configured to be positioned at least proximate to a urethra of a user. While the urine collection device 212 shown in FIG. 2 includes a female urine collection device, the urine collection device 212 may instead include a male urine collection device. PCT International Application No. PCT/US2019/029616, for example, describes various embodiments of both male and female fluid collection devices, the disclosure of which are incorporated in their entirety by this reference. Moreover, the urine collection device 212 may be interchangeable in the urine collection system 200 between different types, varieties, and sizes of male or female urine collection devices or other fluid collection devices. Generally, the urine collection device 212 may include a surface sized to be positioned proximate or adjacent to the urethra and configured to wick urine or other fluids away from the user. Urine or other fluids may be wicked from the surface to a reservoir in the urine collection device 212.

The urine collection system 200 also includes a first conduit 217a in fluid communication with an interior region (e.g. reservoir) of the urine collection device 212 and an interior region of the urine collection container 214. The first conduit 217a may be positioned between the urine collection device 212 and the urine collection container 214. The urine collection system 200 also may include a second conduit 217b providing fluid communication between the pump 216 and the interior region of the urine collection container 214. In some embodiments, the pump 216 may be secured directly to the urine collection container 214, and the second conduit 217b may be absent from the urine collection system 200. The conduits 217a, 217b may include a flexible tube. In some embodiments, at least a portion of the first conduit 217a is substantially opaque, thereby inhibiting viewing of the urine within the first conduit 217a.

The urine collection container 214 may be reusable and dishwasher safe, and may include a generally rigid material such as polycarbonate or glass. In some embodiments, the urine collection container 214 may be disposable. The urine collection system 200 and a container support 302 may be configured to accommodate different volumetric sizes of urine collection containers 214. In some embodiments, a lid 219 having multiple ports and/or adapters for attachment of the conduits 217a, 217b thereto may be secured or securable to the urine collection container 214. Except for the ports and/or adapters for attachment of the conduits 217a, 217b, the urine collection container 214 may be sealed and airtight effective resulting in the technical effect of preventing the urine collected in the urine collection container 214 from leaking or spilling when the wheelchair 300 changes orientation.

The urine collection system also may include a sensor 215. The sensor 215 is secured to the urine collection container 214 and/or the lid 219 and configured to detect a property relating at least to a volume of the urine in the interior region of the urine collection container 214, according to an embodiment. In some embodiments, the sensor 215 includes an ultrasonic sensor, a laser sensor, a wet sensor, a water sensor, a negative pressure sensor, a weight sensor, or an ultraviolet (UV) sensor. In at least one, some, or all of the embodiments of the urine collection system 200, the sensor 215 may be provides the technical effect of a continuous or periodical feedback of the property relating at least to a volume of the urine with or without the sensor 215 touching the urine in the urine collection container 214. The sensor 215 may be secured to the urine collection container 214 or the lid 219 with at least a portion of the sensor 215 inside the interior region of the urine collection container 214. In some embodiments, the sensor 215 is positioned at a top of the urine collection container 214 and pointed downwards where the urine collects in the interior region of the urine collection container 214.

In some embodiments, the sensor 215 includes a weight sensor secured to one of the urine collection container 214 or the container support 302 and configured to detect a weight of the volume of urine collected in the urine collection container 214. In some embodiments, the sensor 215 includes a level transmitter configured to detect a level of the urine in the urine collection container 214. The sensor 215 may include a laser level sensor that measures the distance between the laser transmitter of the sensor 215 to a surface of the urine and back to a detector of the sensor 215. The elapsed time between transmitting the laser and detecting the reflected laser may be measured by the sensor 215 and a distance between the sensor 215 and the level of the urine may be calculated by the sensor 215 or an associated controller. The laser may be transmitted through a clear window on the urine collection container 214 or at least the transmitter of the sensor 215 may be positioned within the urine collection container 214.

In some embodiments, the sensor 215 may include an ultrasonic level sensor that leverages the speed of sound to detect the property relating at least to a volume of the urine in the urine collection container 214. For example, the sensor 215 may measure a distance between a transducer of the sensor 215 and a surface of the urine in the urine collection container 214 using a time for the pulse to travel from the transducer to the surface of the urine and back to the transducer of the sensor 215. The sensor 215 or a controller associated with the sensor 215 may determine a volume of the urine in the urine collection container 214 using the distance between the sensor 214 and the surface of the urine in the urine collection container 214.

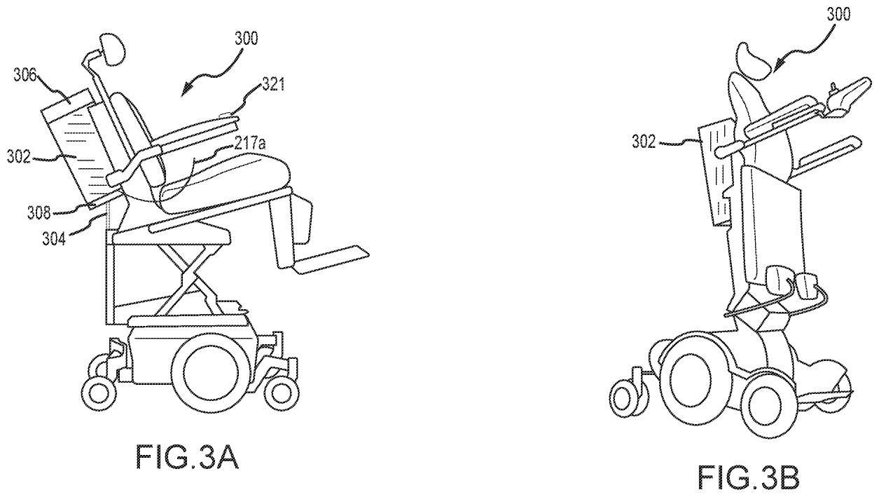

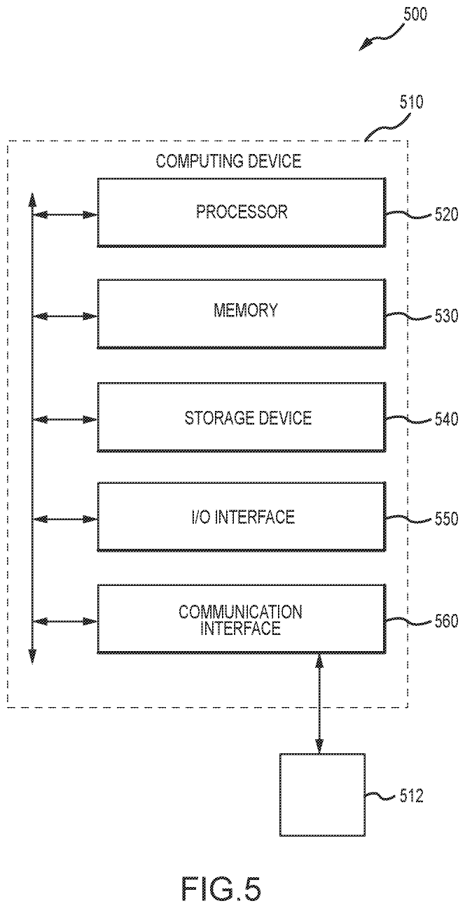

In some embodiments, the sensor 215 also includes a controller. The controller may be included with the sensor 215 or may be a separate component configured to communicate with the sensor 215. For example, the sensor 215 may include a communication interface configured to communicate with a controller 321 (shown in FIG. 3A) mounted on the wheelchair or the container support 302. The controller may include a processor configured to calculate a level or volume of urine in the urine collection container 214. The sensor 215 and/or the controller may include a communication interface configured to send notifications or alerts to other electronic devices. For example, the communication interface may be configured to send notifications or alerts at a selected radio frequency, via BLUETOOTH, or via WI-FI to another electronic device, such as a mobile phone of the user or caregiver. The sensor 215 and/or the controller may be powered by an external or internal battery, such as a rechargeable battery. FIG. 5 provides additional details of a controller 500 that may include the controller of the urine collection system 200.

In at least one, some, or all of the embodiments of the urine collection system 200, the configuration of the controller results in the technical effect of wirelessly transmitting one or more alerts to at least one of an electronic device of the user or a caregiver. For example, the controller may be configured to transmit an alert to an electronic device of the user or a caregiver when the property relating at least to the volume of the urine detected by the sensor 215 indicates the volume of the urine in the urine collection container 215 has reached or exceeded a predetermined volume. For example, based on data from the sensor 215, the controller may wirelessly transmit an alert to an electronic device and/or coordinate an alert to notify the user or the caregiver that the urine in the urine collection device is a predetermined distance (such as about 2.5 cm) from the sensor 215 and/or contacting the sensor 215, and emptying of the urine collection container 214 is recommended. In some embodiments, the controller may wirelessly transmit and/or coordinate alerts and selected frequencies, such as selected time and/or volume intervals. In some embodiments, the controller is configured to coordinate an alert to notify the user or the caregiver that the volume of the urine in the urine collection container 214 has reached or exceeded two or more of 50% of a maximum volume of the urine collection container, 75% of the maximum volume of the urine collection container, and/or 90% of the maximum volume of the urine collection container.

In some embodiments, the controller may wirelessly transmit an alert to the electronic device of the user or the caregiver and/or coordinate a battery charging alert to notify the user or the caregiver that charging of the rechargeable battery powering at least one of the pump 216 or the controller is suggested. The controller may wirelessly transmit an alert to the electronic device of the user or the caregiver and/or coordinate an alert to notify the user or the caregiver when replacement of a filter (such as the filter 218) of the urine collection system 200 is recommended. In some embodiments, the controller may wirelessly transmit a solar panel alert to the electronic device of the user or the caregiver and/or coordinate a solar panel alert indicating performance of the solar panel. In some embodiments, the controller may wirelessly transmit a position alert to the electronic device of the user or the caregiver and/or coordinate a position alert indicating a change of position by the user is suggested. Alerts coordinated by the controller may include one or more of a light, a noise, and/or a haptic feedback.

In at least one, some, or all of the embodiments of the urine collection system 200, a filter 218 is included that results in the technical effect of neutralizing and/or enhancing (e.g., improving) odor of the air being pulled from the interior region of the urine collection container 214 by the pump 216. In some embodiments, the filter 218 is positioned on an exhaust vent on the pump 216. In some embodiments, the filter 218 is positioned between at least a portion of the conduit 217b and a portion of the interior region of the urine collection container 214 such that air being pulled from the interior region of the urine collection container 214 is filtered before or as the air enters the conduit 217b. In some embodiments, a filter 218 is positioned both at the exhaust vent on the pump 216 and before air enters the conduit 217b. In some embodiments, the filter 218 may be positioned at least partially within the second conduit 217b and/or throughout one or more portions of the second conduit 217b. The filter 218 may include an odor absorbing filter, such as activated carbon and/or zeolite filters.

In some embodiments, the filter 218 may include an aromatherapy pack or an aromatherapy pack may be secured proximate to the exhaust vent of the pump to produce a more pleasant smell. The filter 218 may include baking soda or other composition that removes odor from the air and/or adds pleasant aroma to the air. In some embodiments, the filter 218 is absent and the pump 216 includes the aromatherapy pack removably secured to the pump 216 proximate to the exhaust vent.

In at least one, some, or all embodiments of the urine collection system 200, the pump 216 is in fluid communication with the interior region of the urine collection container 214 and is configured to pull a vacuum on the interior region of the urine collection container 214 effective to draw the urine from the urine collection device 212 through the first conduit 217a into the urine collection container 214. The pump 216 may be secured directly to the urine collection container 214, or the conduit 217b may fluidly couple the pump 216 with the interior region of the urine collection container.

The pump 216 may include one or more of a manual vacuum pump, and electric vacuum pump, a diaphragm pump, a centrifugal pump, a displacement pump, a magnetically driven pump, a peristaltic pump, or any pump configured to produce a vacuum. The pump 216 may provide a vacuum or suction to remove fluid from the fluid collection device 212. In some examples, the pump 216 may be powered by one or more batteries or other power sources. In some examples, the pump 216 may be sized and shaped to fit within the container support 302. The pump 216 may include one or more of a switch, a button, a plug, a remote, or any other device suitable to activate the pump 216.

FIGS. 3A and 3B show a container support 302 of a urine collection system mounted to a wheelchair 300. The container support 302 is configured to be mounted to a motorized wheelchair 300 and support the urine collection container 214 and/or the pump 216 therein when secured to the motorized wheelchair 300. The container support 302 includes one or more rigid walls. For example, the container support 302 may include at least a rigid bottom wall configured to support at least the urine collection container 214. In some embodiments, the container support 302 includes one or more side walls that also are generally rigid. In some embodiments, the container support 302 includes one or more side walls that are generally flexible. For example, the container support 302 may include one more side walls that are generally flexible and/or adjustable effective to allow an internal volume of the container support to be adjusted. Rigid walls of the container support 302 may include plastic, metal, word, composite materials, or combinations thereof. In some embodiments, rigid walls of the container support 302 may include a lining material or fabric.

The container support 302 may include a top wall or lid that is generally rigid or flexible. In some embodiments, the top wall or lid is adjustable between an open position and a closed position. For example, in an open position, the top wall or lid may be connected to a sidewall via one or more hinges or connecting material. When the top wall or lid is in the open position, a user or caregiver may insert and remove the urine collection container 214 and/or the pump into the container support 302. The top wall or lid may include a locking mechanism such as a latch, clamp, clip, or zipper configured to selectively retain the top wall or lid in the closed position.

In at least one, some, or all embodiments, the container support 302 includes one or more sound dampening elements such as foam and/or sound dampening fabric on the interior surface of one or more walls of the container support 302 resulting in the technical effect of reducing or eliminating operational noises (such as the pump 216 running and/or splashing of urine) of the urine collection system 200 emanating from the container support 302. In at least one, some, or all embodiments, the container support 302 may include a window in a sidewall or the top wall resulting in the technical effect of allowing a user or caregiver to view a urine level in the urine collection container 214. The sidewalls may otherwise be opaque effective to obscure undesirable viewing of the urine collection container 214 housed in the container support 302.

The container support 302 may be mounted to a motorized wheelchair 300 that is adjustable between a standing orientation and a sitting orientation. FIG. 3A shows the wheelchair 300 in the sitting orientation and FIG. 3B shows the wheelchair in the standing orientation. In some embodiments, the container support 302 may be mounted to a motorized wheelchair 300 that is adjustable between a standing orientation, a sitting orientation, and/or a reclined or lay flat orientation. When the pump 216 and urine collection container 214 are positioned within the container support 302, the urine collection system 200 may collect and temporarily store urine from a user of the wheelchair 300 when the wheelchair 300 is in any of the standing orientation, the sitting orientation, and/or the reclined orientation. A urine collection system that is mountable to a wheelchair 300 having a standing orientation is beneficial because standing reduces pressure on internal organs of the user and improves lung volume, which increases blood oxygenation in the user. Standing also reduces edema, the risk of deep vein thrombosis, and the risk of urinary tract infections in the user

The container support 302 may be fixedly or detachably mounted or secured to the back or underneath the seat of the wheelchair 300. In some embodiments, the container support 302 is mechanically mounted to wheelchair 300 with one or more fasteners, straps, support arms, clamps, clips, or any combinations thereof. The container support 302 is sized and dimensioned to house and/or support the urine collection container 214 and the pump 216 therein. The container support 302 may be sized and dimensioned to house and/or support urine collection containers and pumps of other urine collection systems therein. The container support 302 may include a hole or opening in the top or a sidewall sized and dimensioned to extend a portion of the first conduit 217a through the container support 302 to allow urine to be transferred from the urine collection device outside the container support 302 to the urine collection container 214 inside the container support 302. In some embodiments, the container support 302 includes a port or adapter configured to attach or secure to the first conduit 217a. The urine collection system 200 also may include an additional conduit positioned in the container support 302 may provide fluid communication between the first conduit 217a and the urine collection container 214. Accordingly, the first conduit 217a may attach to the port or adapter on the container support 302 on the outside of the container support 302 and the additional conduit may attach to the port or adapter on the container support on the inside of the container support 302, with the port or adapter providing fluid communication between the first conduit 217a and the additional conduit housed in the container support 302.

In some embodiments, the urine collection system 200 may include a rechargeable battery 308 electrically connected to the pump 216 and configured to power the pump 216. The rechargeable battery 308 may be removably mounted or secured to the container support 302. In some embodiments, the rechargeable battery 308 may be positioned within the container support 308 and not visible outside the container support 302. For example, the rechargeable battery 308 may be mounted within the container support 302 or mounted to the pump 216. In some embodiments, the rechargeable battery 308 may be electrically coupled to the battery or power source of the wheelchair 300 with an electrical cable 304. The electrical cable 304 also may be configured to connect to a wall outlet for charging the rechargeable battery. In some embodiments, the electrical cable 304 electrically couples the pump 216 with the battery or power source of the wheelchair 300.

In some embodiments, the container support 302 includes one or more solar panels 306 secured to an outer portion of the container support 302 and electrically coupled to at least one of the rechargeable battery 308 and/or the pump 216. The one or more solar panels 306 may be configured to at least partially recharge the rechargeable battery 308 and/or power the pump 216. In some embodiments, a controller 321 may be secured to the arm or other member of the wheelchair 300. The controller 321 may include a touch screen and may include any aspect of the controller described above in relation to the urine collection system 200.

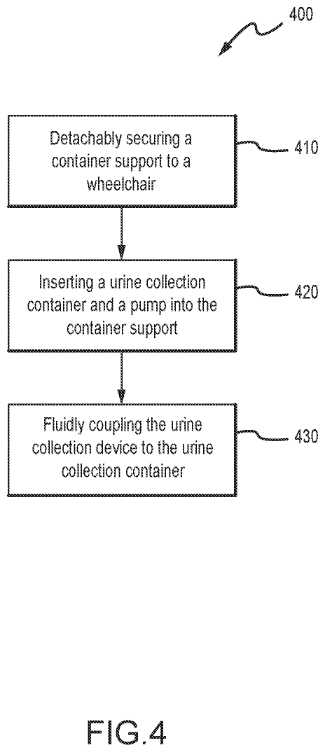

FIG. 4 is a flow diagram of a method 400 for assembling a portable urine collection system, according to an embodiment. The method includes an act 410 of detachably securing a container support to a motorized wheelchair that is adjustable between a sitting orientation, a standing orientation, and a reclined orientation. In some embodiments, the act 410 includes detachably securing the container support to a back of the motorized wheelchair. In some embodiments, the act 410 includes detachably securing the container support underneath a seat of the wheelchair.

The method 400 may include an act 420 of inserting a urine collection container and a pump in fluid communication with an interior region of the urine collection container in the container support. In some embodiments, the method 400 also includes an act inserting one or more sound dampening elements into the container support before or after the urine collection container and the pump are inserted into the container support. In some embodiments, the method 400 also includes an act of positioning a urine collection device proximate to a urethra of a user.

The method 400 may include an act 430 of fluidly coupling the urine collection device to the urine collection container with a first conduit. The pump may be configured to pull a vacuum on the interior region of the urine collection container effective to draw urine from the urine collection device through the first conduit into the urine collection container and draw urine from the urine collection device when the motorized wheelchair is in each of the sitting orientation, the standing orientation, and the reclined orientation.

In some embodiments, the method 400 also includes an act of electrically coupling a solar panel on the outer portion of the container support to a rechargeable battery that is electrically coupled to the pump. The method may include electrically coupling the rechargeable battery to one or more of a power source of the motorized wheelchair or an electrical outlet with a cable to recharge the rechargeable battery or power the pump. In some embodiments, the method includes electrically coupling the pump to a battery or power source of the motorized wheelchair with a cable to power the pump.

In some embodiments, the method 400 includes an act of activating a sensor secured to one of the urine collection container or the container support to detect a property relating at least to a volume of the urine in the urine collection container. The method 400 may include an act of activating a controller to communicate with the sensor and coordinate a user alert when the volume of the urine in the urine collection container has reached or exceeded a predetermined volume. The controller may be configured coordinate a user alert to notify the user or a caregiver that the volume of the urine in the urine collection container has reached or exceeded the predetermined volume. The method 400 may include an act of activating the controller to coordinate the user alert to notify the user or the caregiver that the volume of the urine in the urine collection container has reached or exceeded two or more of 50% of a maximum volume of the urine collection container, 75% of the maximum volume of the urine collection container, or 90% of the maximum volume of the urine collection container. The method 400 may include an act of activating the controller to coordinate at least one of a filter replacement alert indicating replacement of the one or more activated carbon and zeolite filters is suggested, a battery charging alert indicating charging of the rechargeable battery is suggested, a battery replacement alert indicating replacement of the rechargeable battery is suggested, a solar panel alert indicating performance of the solar panel, or a position alert indicating a change of position by the user is suggested. In some embodiments, the method includes securing the controller to an arm of the motorized wheelchair.

The acts of the method 400 described above are for illustrative purposes. For example, the acts of the method 400 can be performed in different orders, split into multiple acts, modified, supplemented, or combined. In an embodiment, one or more of the act of the method 400 can be omitted from the method 400. Any of the acts of the method 400 can include using any of the portable urine collection systems disclosed herein.

FIG. 5 is a schematic of a controller 500 that may be used with any of the systems and methods described herein, according to an embodiment. For example, the controller of the urine collection system 200 and/or the controller 321 may include any aspect of the controller 500. The controller 500 may be configured to implement any of the example acts or steps disclosed herein, such as wired or wirelessly communicating with the sensor 215 or other electronic devices, determining a volume of the urine in the urine collection container 214, and/or transmitting and/or coordinating alerts described herein.

The controller 500 includes at least one computing device 510, according to an embodiment. The at least one computing device 510 is an exemplary computing device that may be configured to perform one or more of the acts described above. The computing device 510 can comprise at least one processor 520, memory 530, a storage device 540, an input/output (“I/O”) device/interface 550, and a communication interface 560. While an example computing device 510 is shown in FIG. 5, the components illustrated in FIG. 5 are not intended to be limiting of the controller 500 or computing device 510. Additional or alternative components may be used in some examples. Further, in some examples, the controller 500 or the computing device 510 can include fewer components than those shown in FIG. 5. For example, the controller 500 may not include the one or more additional computing devices 512. In some examples, the at least one computing device 510 may include a plurality of computing devices. Components of computing device 510 shown in FIG. 5 are described in additional detail below.

In some examples, the processor(s) 520 includes hardware for executing instructions (e.g., instructions for carrying out one or more portions of any of the methods disclosed herein), such as those making up a computer program. For example, to execute instructions, the processor(s) 520 may retrieve (or fetch) the instructions from an internal register, an internal cache, the memory 530, or a storage device 540 and decode and execute them. In particular examples, processor(s) 520 may include one or more internal caches for data such as tables pertaining to volumetric tables. As an example, the processor(s) 520 may include one or more instruction caches, one or more data caches, and one or more translation lookaside buffers (TLBs). Instructions in the instruction caches may be copies of instructions in memory 530 or storage device 540. In some examples, the processor 520 may be configured (e.g., include programming stored thereon or executed thereby) to carry out one or more portions of any of the example methods disclosed herein.

In some examples, the processor 520 is configured to perform any of the acts disclosed herein or cause one or more portions of the computing device 510 or controller 500 to perform at least one of the acts disclosed herein. Such configuration can include one or more operational programs (e.g., computer program products) that are executable by the at least one processor 520. For example, the processor 520 may be configured to automatically determine a volume of urine in a urine collection container, automatically determine a proximity of urine in the urine collection container to a sensor, automatically transmit an alert when the volume of the urine in the urine collection container meets or exceeds a predetermined threshold, automatically transmit an alert when a change of filter is suggested, and/or automatically transmit an alert when a change or recharge of battery is suggested.

The at least one computing device 510 (e.g., a server) may include at least one memory storage medium (e.g., memory 530 and/or storage device 540). The computing device 510 may include memory 530, which is operably coupled to the processor(s) 520. The memory 530 may be used for storing data, metadata, and programs for execution by the processor(s) 520. The memory 530 may include one or more of volatile and non-volatile memories, such as Random Access Memory (RAM), Read Only Memory (ROM), a solid state disk (SSD), Flash, Phase Change Memory (PCM), or other types of data storage. The memory 530 may be internal or distributed memory.

The computing device 510 may include the storage device 540 having storage for storing data or instructions. The storage device 540 may be operably coupled to the at least one processor 520. In some examples, the storage device 540 can comprise a non-transitory memory storage medium, such as any of those described above. The storage device 540 (e.g., non-transitory storage medium) may include a hard disk drive (HDD), a floppy disk drive, flash memory, an optical disc, a magneto-optical disc, magnetic tape, or a Universal Serial Bus (USB) drive or a combination of two or more of these. Storage device 540 may include removable or non-removable (or fixed) media. Storage device 540 may be internal or external to the computing device 510. In some examples, storage device 540 may include non-volatile, solid-state memory. In some examples, storage device 540 may include read-only memory (ROM). Where appropriate, this ROM may be mask programmed ROM, programmable ROM (PROM), erasable PROM (EPROM), electrically erasable PROM (EEPROM), electrically alterable ROM (EAROM), or flash memory or a combination of two or more of these. In some examples, one or more portions of the memory 530 and/or storage device 540 (e.g., memory storage medium(s)) may store one or more databases thereon.

In some examples, one or more of a history of the volume of the urine in the urine collection container, a trend of the volume of the urine in the urine collection container, a history of filter replacement, and/or a history of battery replacement or recharging may be stored in a memory storage medium such as one or more of the at least one processor 520 (e.g., internal cache of the processor), memory 530, or the storage device 540. In some examples, the at least one processor 520 may be configured to access (e.g., via bus 570) the memory storage medium(s) such as one or more of the memory 530 or the storage device 540. For example, the at least one processor 520 may receive and store the data (e.g., look-up tables) as a plurality of data points in the memory storage medium(s). The at least one processor 520 may execute programming stored therein adapted access the data in the memory storage medium(s) to automatically determine a volume of urine in a urine collection container, automatically determine a proximity of urine in the urine collection container to a sensor, automatically transmit an alert when the volume of the urine in the urine collection container meets or exceeds a predetermined threshold, automatically transmit an alert when a change of filter is suggested, and/or automatically transmit an alert when a change or recharge of battery is suggested. For example, the at least one processor 520 may access one or more look-up tables in the memory storage medium(s) such as memory 530 or storage device 540.

The computing device 510 also includes one or more I/O devices/interfaces 550, which are provided to allow a user to provide input to, receive output from, and otherwise transfer data to and from the computing device 510. These I/O devices/interfaces 550 may include a mouse, keypad or a keyboard, a touch screen, camera, optical scanner, network interface, web-based access, modem, a port, other known I/O devices or a combination of such I/O devices/interfaces 550. The touch screen may be activated with a stylus or a finger.

The I/O devices/interfaces 550 may include one or more devices for presenting output to a user, including, but not limited to, a graphics engine, a display (e.g., a display screen or monitor), one or more output drivers (e.g., display drivers), one or more audio speakers, and one or more audio drivers. In certain examples, I/O devices/interfaces 550 are configured to provide graphical data to a display for presentation to a user. The graphical data may be representative of one or more graphical user interfaces and/or any other graphical content as may serve a particular implementation.

The computing device 510 can further include a communication interface 560. The communication interface 560 can include hardware, software, or both. The communication interface 560 can provide one or more interfaces for communication (such as, for example, packet-based communication) between the computing device 510 and one or more additional computing devices 512 or one or more networks. For example, communication interface 560 may include a network interface controller (NIC) or network adapter for communicating with an Ethernet or other wire-based network or a wireless NIC (WNIC) or wireless adapter for communicating with a wireless network, such as a WI-FI. The one or more additional computer device 512 may include a smart phone of the user, a smart phone of the caregiver, an electronic device on the wheelchair, and/or a computer device of a healthcare system.

Any suitable network and any suitable communication interface 560 may be used. For example, computing device 510 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, one or more portions of controller 500 or computing device 510 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination thereof. The computing device 510 may include any suitable communication interface 560 for any of these networks, where appropriate.

The computing device 510 may include a bus 570. The bus 570 can include hardware, software, or both that couples components of computing device 510 to each other. For example, bus 570 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination thereof.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

What is claimed is:

1. A portable urine collection system, comprising:
a urine collection device configured to be positioned at least proximate to a urethra of a user;
a first conduit in fluid communication with the urine collection device;
a urine collection container having an interior region;
a pump in fluid communication with the urine collection container and configured to pull an at least partial vacuum on the interior region of the urine collection container effective to draw urine from the urine collection device through the first conduit into the urine collection container; and
a container support configured to detachably secure to a motorized wheelchair that is adjustable between a sitting orientation, a standing orientation, and a reclined orientation, wherein the container support is sized and dimensioned to support the urine collection container and the pump therein, and the pump is configured to draw the urine from the urine collection device when the motorized wheelchair is in each of the sitting orientation, the standing orientation, and the reclined orientation.

2. The portable urine collection system of claim 1, wherein the container support includes one or more sound dampening elements.

3. The portable urine collection system of any of claims 1-2, wherein the container support is configured to mechanically secure to a back of the motorized wheelchair or underneath a seat of the motorized wheelchair.

4. The portable urine collection system of any of claims 1-3, further comprising:
a rechargeable battery electrically coupled to the pump and configured to power the pump; and
a solar panel secured to an outer portion of the container support and electrically coupled to the rechargeable battery, the solar panel configured to at least partially recharge the rechargeable battery.

5. The portable urine collection system of claim 4, further comprising a cable electrically coupled to the rechargeable battery and configured to couple to one or more of a power source of the motorized wheelchair or an electrical outlet to recharge the rechargeable battery or power the pump.

6. The portable urine collection system of any of claims 1-3, further comprising a cable electrically coupled to the pump and configured to couple to a battery or a power source of the motorized wheelchair to power the pump.

7. The portable urine collection system of any of claims 1-6, further comprising:
a second conduit providing fluid communication between the urine collection container and the pump; and
one or more activated carbon and zeolite filters configured to remove odors from air passing therethrough, the one or more activated carbons being positioned in the second conduit, between the interior region of the urine collection container and the second conduit, or at least proximate to an exhaust of the pump.

8. The portable urine collection system of any of claims 1-7, further comprising a sensor secured to one of the urine collection container or the container support and configured to detect a property relating at least to a volume of the urine in the urine collection container.

9. The portable urine collection system of claim 8, wherein the sensor includes a weight sensor secured to one of the urine collection container or the container support and configured to detect a weight of the volume of the urine collected in the urine collection container.

10. The portable urine collection system of claim 9, wherein the sensor includes a level sensor secured to one of the urine collection container or the container support and configured to detect a level of the volume of the urine collected in the urine collection container.

11. The portable urine collection system of any of claims 8-10, further comprising a controller configured to communicate with the sensor, wherein the sensor is configured communicate an alert to the controller when the volume of the urine in the urine collection container has reached or exceeded a predetermined volume and the controller is configured to coordinate a user alert to notify the user or a caregiver that the volume of the urine in the urine collection container has reached or exceeded the predetermined volume.

12. The portable urine collection system of claim 11, wherein the user alert coordinated by the controller includes at least one of a light, a noise, or a haptic feedback.

13. The portable urine collection system of any of claims 11-12, wherein the controller is configured to coordinate the user alert to notify the user or the caregiver that the volume of the urine in the urine collection container has reached or exceeded two or more of 50% of a maximum volume of the urine collection container, 75% of the maximum volume of the urine collection container, and/or 90% of the maximum volume of the urine collection container.

14. The portable urine collection system of any of claims 11-13, wherein the controller is configured to coordinate at least one of a filter replacement alert indicating replacement of the one or more activated carbon and zeolite filters is suggested, a battery charging alert indicating charging of the rechargeable battery is suggested, a battery replacement alert indicating replacement of the rechargeable battery is suggested, a solar panel alert indicating performance of the solar panel, or a position alert indicating a change of position by the user is suggested.

15. The portable urine collection system of any of claims 11-14, wherein the controller is secured to an arm of the motorized wheelchair and includes a touch screen.

16. A method of assembling a portable urine collection system, the method comprising:
detachably securing a container support to a motorized wheelchair that is adjustable between a sitting orientation, a standing orientation, and a reclined orientation;
inserting a urine collection container and a pump in fluid communication with an interior region of the urine collection container in the container support;
positioning a urine collection device proximate to a urethra of a user; and
fluidly coupling the urine collection device to the urine collection container with a first conduit, wherein the pump is configured to pull an at least partial vacuum on the interior region of the urine collection container effective to draw urine from the urine collection device through the first conduit into the urine collection container and draw urine from the urine collection device when the motorized wheelchair is in each of the sitting orientation, the standing orientation, and the reclined orientation.

17. The method of claim 16, further comprising inserting one or more sound dampening elements into the container support.

18. The method of any of claim 16 or 17, wherein detachably securing a container support to a motorized wheelchair includes detachably securing the container support to a back of the motorized wheelchair.

19. The method of any of claim 16 or 17, wherein detachably securing a container support to a motorized wheelchair includes detachably securing the container support underneath a seat of the wheelchair.

20. The method of any of claims 16-19, further comprising electrically coupling a solar panel on the outer portion of the container support to a rechargeable battery that is electrically coupled to the pump.

21. The method of claim 20, further comprising electrically coupling the rechargeable battery to one or more of a power source of the motorized wheelchair or an electrical outlet with a cable to recharge the rechargeable battery or power the pump.

22. The method of any of claims 16-18, further comprising electrically coupling the pump to a battery or power source of the motorized wheelchair with a cable to power the pump.

23. The method of any of claims 16-22, further comprising activating a sensor secured to one of the urine collection container or the container support to detect a property relating at least to a volume of the urine in the urine collection container.

24. The method of claim 23, further comprising activating a controller to communicate with the sensor and coordinate a user alert when the volume of the urine in the urine collection container has reached or exceeded a predetermined volume and the controller is configured to coordinate a user alert to notify the user or a caregiver that the volume of the urine in the urine collection container has reached or exceeded the predetermined volume.

25. The method of claim 24, further comprising activating the controller to coordinate the user alert to notify the user or the caregiver that the volume of the urine in the urine collection container has reached or exceeded two or more of 50% of a maximum volume of the urine collection container, 75% of the maximum volume of the urine collection container, and/or 90% of the maximum volume of the urine collection container.

26. The method of any of claim 24 or 25, further comprising activating the controller to coordinate at least one of a filter replacement alert indicating replacement of the one or more activated carbon and zeolite filters is suggested, a battery charging alert indicating charging of the rechargeable battery is suggested, a battery replacement alert indicating replacement of the rechargeable battery is suggested, a solar panel alert indicating performance of the solar panel, or a position alert indicating a change of position by the user is suggested.

27. The method of any of claims 24-26, further comprising securing the controller to an arm of the motorized wheelchair.
